(19) United States
(12) Patent Application Publication
(10) Pub. No.: US 2020/0197298 A1
(43) Pub. Date: Jun. 25, 2020

(54) Apparatus and Methods for Delivering Volatilized Compositions Having Pharmacological Effects

(71) Applicant: Chris Lindsay, Clackamas, OR (US)

(72) Inventor: Chris Lindsay, Clackamas, OR (US)

(21) Appl. No.: 16/571,408

(22) Filed: Sep. 16, 2019

Related U.S. Application Data
(60) Provisional application No. 62/782,410, filed on Dec. 20, 2018.

(51) Int. Cl. A61K 9/00; A61K 31/352; A61M 11/04; A61K 31/047; A61K 47/44

(52) U.S. Cl. CPC A61K 9/0073; A61K 31/352; A61K 47/44; A61K 31/047; A61M 11/04

(57) ABSTRACT

Apparatus and method for delivering cannabinoids such as CBD to users are disclosed. A source of a cannabinoid of interest is combined with a blend of herbs in an aromatherapy wrap garment. The user heats the garment to thereby volatilize the cannabinoid and the essential oils in the herbs. The volatilized oils flow through the fabric garment and are both inhaled by the user and absorbed transdermally. Heat retention ingredients such as flax seed or other grains, including but not limited to rice, barley and other materials such as clay or plastic beads may be included in the garment.

### TECHNICAL FIELD

The present invention relates generally to apparatus used in the field of aromatherapy for delivering essential oils for pharmacological and therapeutic effects, and more specifically, to aromatherapy wraps that incorporate materials that include cannabis and/or compounds derived from cannabis, including cannabinoids such as cannabidiol (“CBD”) and other compounds.

### BACKGROUND

Aromatherapy has been utilized for many years as a holistic healing treatment that uses natural plant extracts to promote health and well-being. Sometimes aromatherapy is called essential oil therapy because it uses aromatic essential oils medicinally to improve the health of the body, mind, and spirit; it enhances both physical and emotional health. Aromatherapy is thought of as both an art and a science and recently, aromatherapy has gained more recognition in the fields of science and medicine.

Aromatherapy generally uses essential oils released from phyto-derived (i.e., plant-derived) materials, such as the leaves of plants, for therapeutic benefits. By heating the phyto-derived materials to a temperature sufficient for vaporization, essential oils and extracts may be emitted from the plant material as vapor and the vapor may be inhaled by a user for its therapeutic benefits.

There are a number of known devices for vaporizing the essential oils in plant materials for aromatherapy. One such device of particular interest herein is commonly called an “aromatherapy wrap.” In short, an aromatherapy wrap is a garment-like device that is packed with blends of herbs and other plants that contain the essential oils that are intended to be vaporized and inhaled. The fabric materials that are used to make the wrap are permeable to allow the vaporized essential oils to be released during use. The user heats the wrap (for instance, in a microwave oven) to heat the phyto-derived materials in the wrap to a temperature sufficient to vaporize the essential oils. The user then wears the heated wrap and inhales the vaporized essential oils that are released through the wrap for their therapeutic effects.

There are many types of aromatherapy wraps and the applicant for and assignee of the present provisional patent application manufactures and sells a selection of wraps at, for instance, www.herbalconcepts.com and www.kozizones.com. In the wraps that are made and sold by the applicant herein, flax, wheat or other grains are incorporated in the blend of herbs as heat retention sources. In a normal microwave used in residential settings, 1-2 minutes of time in the microwave oven will heat the contents of the wrap to around 130 degrees and cause vaporization of the essential oils.

A typical formulation of phyto-materials used in an aromatherapy wrap is as follows:

| Ingredient, common name | Ingredient, botanical name |
|---|---|
| lavender | lavendula angustifolia |
| spearmint | mentha spicata |
| Peppermint | mentha piperita |
| Valerian | valerian officinalis |
| Yarrow | achillea millefoliurn |
| Cinnamon | cinnamonum verurn |
| Rosemary | rosmarinus officinalis |
| Chamomile | anthemis nobilis |
| Lemon grass | cymbopogon citrates |
| White willow | salix alba |
| Yellow dock | rumex crispus |
| Hops | humulus lupulus |

The herbs listed above are exemplary only and there are many other phyto-derived materials that may be utilized in aromatherapy. Moreover, the percentage of each ingredient may be varied according to the types of essential oils that are desired in a specific product, and the percentage of each ingredient in the blend of herbs may be varied. As noted, a heat-retaining grain such as flax seed is incorporated in the blend of herbs to encourage heating of the herbs to volatilize the essential oils.

In recent years many states have passed legislation that decriminalizes consumption of cannabis and cannabis derivatives for recreational use. Even more states have decriminalized cannabis use for medical purposes. As a result, there is increasing interest in and demand for cannabis and cannabis derivative products in many states. There are many known cannabinoids that may be derived from cannabis and hemp plants. One such derivative is the cannabinoid commonly called “CBD”—cannabidiol. CBD use for its therapeutic effects is gaining widespread acceptance and interest in use of CBD and other cannabis derivatives is increasing rapidly. CBD is commonly available in oil form, ointments, creams and lotions, patches, balms, and salves. The CBD from these products is readily absorbed into the skin and can be applied directly to affected areas. The oils are also versatile, as they may be applied topically or ingested. CBD topical oils are known to be an effective treatment for many symptoms, including having significant potential for pain relief. Anecdotal evidence suggests that CBD may be used to help manage chronic pain in many cases. And CBD oil is especially promising due to its lack of intoxicating effects and a possible lower potential for side effects than many other pain medications. Moreover, unlike THC, which is illegal in many states and under current federal laws, CBD is legal in most states and, depending on several factors, may not be subject to restrictions on interstate commerce.

### SUMMARY OF THE INVENTION

In view of the significant interest in use of cannabinoids such as CBD and other cannabis and hemp-derived products, there is a growing need for apparatus and methods for delivery of the products to users seeking their benefits.

The present invention defines an apparatus and method for delivering cannabinoids such as CBD to users. More specifically, a source of the cannabinoid of interest is combined with a blend of herbs in an aromatherapy wrap garment. The user heats the garment in a microwave oven, thereby volatilizing the cannabinoid and the essential oils in the herbs. The volatilized oils flow through the fabric garment and are both inhaled by the user and absorbed transdermally through the user's skin. Heat retention ingredients such as flax seed or other grains, including but not limited to rice, barley and other materials such as clay or plastic beads may be included in the garment.

### BRIEF DESCRIPTION OF THE DRAWINGS

The invention will be better understood and its numerous objects and advantages will be apparent by reference to the following detailed description of the invention when taken in conjunction with the following drawings.

FIG. 1 is a perspective view of one exemplary aromatherapy wrap garment of the type that may be utilized in accordance with the present invention, namely, a neck pillow, also known as a neck and shoulder wrap.

FIG. 2 is a front elevation view of the aromatherapy wrap garment shown in FIG. 1.

FIG. 3 is a rear elevation view of the aromatherapy wrap garment shown in FIG. 1.

### DETAILED DESCRIPTION OF ILLUSTRATED EMBODIMENTS

The present invention is defined by an aromatherapy wrap garment that incorporates a blend of herbs and a source of cannabinoid compounds and more particularly CBD.

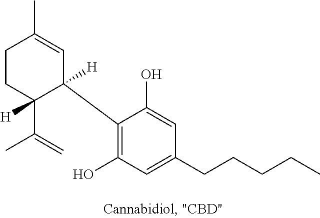 [SMILES: C=C(C)[C@@H]1CCC(C)=C[C@H]1c1c(O)cc(CCCCC)cc1O]

In one embodiment, the source of cannabinoid compounds may be designed for release of tetrahydrocannabinol (“THC”), the principal psychoactive constituent of cannabis.

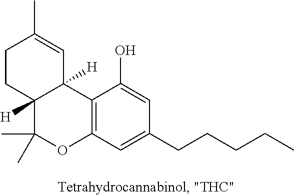 [SMILES: CCCCCc1cc(O)c2c(c1)OC(C)(C)[C@@H]1CCC(C)=C[C@@H]21]

In other embodiments, the source of cannabis derived products may include both CBD and THC.

In a preferred embodiment, the source of cannabinoids for use in the present invention is dehydrated cannabis that has been selected for its cannabinoid content. The cannabinoids used in the present invention may also be sourced from hemp. There is a variety of cannabis plants used for CBD production and there are varieties that are low in THC and high in CBD. If the THC content is too high, that specific variety may not be useful as a CBD product. In still other instances where THC content is desired, other cannabis varieties that have higher levels of THC may be utilized. In an exemplary embodiment, the dehydrated cannabis that is selected may be blended into the other dehydrated herbs such as follows:

| Ingredient, common name | Ingredient, botanical name | Weight Percent |
|---|---|---|
| Lavender | lavendula angustifolia | 9.00 |
| Spearmint | mentha spicata | 3.00 |
| Peppermint | mentha piperita | 3.00 |
| Valerian | valerian officinalis | 0.03 |
| Yarrow | achillea millefoliurn | 0.03 |
| Cinnamon | cinnamonurn verurn | 1.50 |
| Rosemary | rosmarinus officinalis | 4.00 |
| Chamomile | anthemis nobilis | 8.00 |
| Lemon grass | cymbopogon citrates | 3.00 |
| White willow | salix alba | 0.03 |
| Yellow dock | rumex crispus | 0.03 |
| Hops | humulus lupulus | 0.03 |
| Cannabis or cannabis | e.g., cannabis sativa or | 68.35 |
| derivative compounds | cannabis indica | |

It will be recognized that the weight percentages specified in the foregoing table are exemplary only and that the amount of each ingredient may be varied according as desired. The quantity of cannabis formulated into the blend will depend upon the amount of oil in the dehydrated cannabis source material and on other factors such as the specific cannabinoid that is being delivered, such as CBD or THC. In the embodiment exemplified in the foregoing table, the cannabinoid source material is specified in the dehydrated form of the plant. The total weight of a blend of herbs and cannabinoid according to the present invention that incorporated into an aromatherapy wrap according to the invention depends upon the size of the garment—a larger garment contains a greater amount of the blend than a smaller garment. Typically, the weight amount of the non-cannabinoid phyto material in the blend is in the range of between about 1 to 6 grams but this depends on the total weight of the blend in the garment. Typically, the weight percentage of the cannabinoid source material is in the range of between 65 and 75% of the blend. Also taken into consideration in determining the amount of dried source material for the cannabinoid to be added to a blend is the concentration of CBDA in the source material. Typically, dehydrated hemp source material contains between about 10 to 20% CBDA (by weight). Accordingly, such source material is added in an amount sufficient so that the aromatherapy garment contains between about 100 and 600 mg of CBD that can be delivered to the user.

In some embodiments, CBD may be added to the dried herbs by applying a CBD oil or extract to the herbs. The same may be done with oils containing THC. It will be recognized that where the source material for the cannabinoid incorporated into an aromatherapy garment is other than dehydrated phyto material, for instance, a prepared CBD oil extract, the amount of oil extract that is added to the blend of herbs is specified in an amount sufficient to deliver from about 100 and 600 mg CBD to the user.

In some cases the cannabis source material may have an odor that is unpleasant to some users. Unpleasant odors may be alleviated to a degree by masking with the odors from the other herbs, which are commonly taken as being more pleasant, or the unpleasant odors may be alleviated by deodorization of the cannabis material.

Typically, the raw dried hemp plant or hemp biomass (or other CBD-source material) contains CBD in the form of cannabidiolic acid, known as CBDA. CBDA is converted to CBD through decarboxylation by heating the source material to about 158 degrees F.

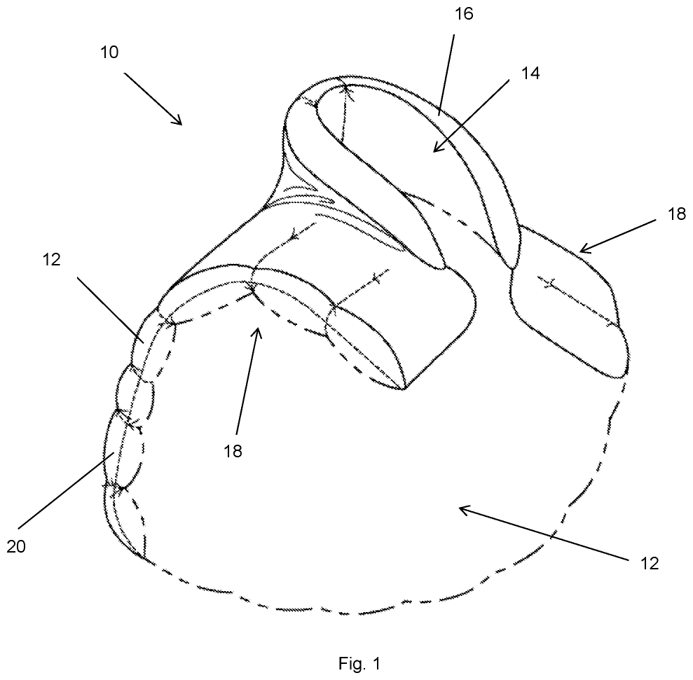

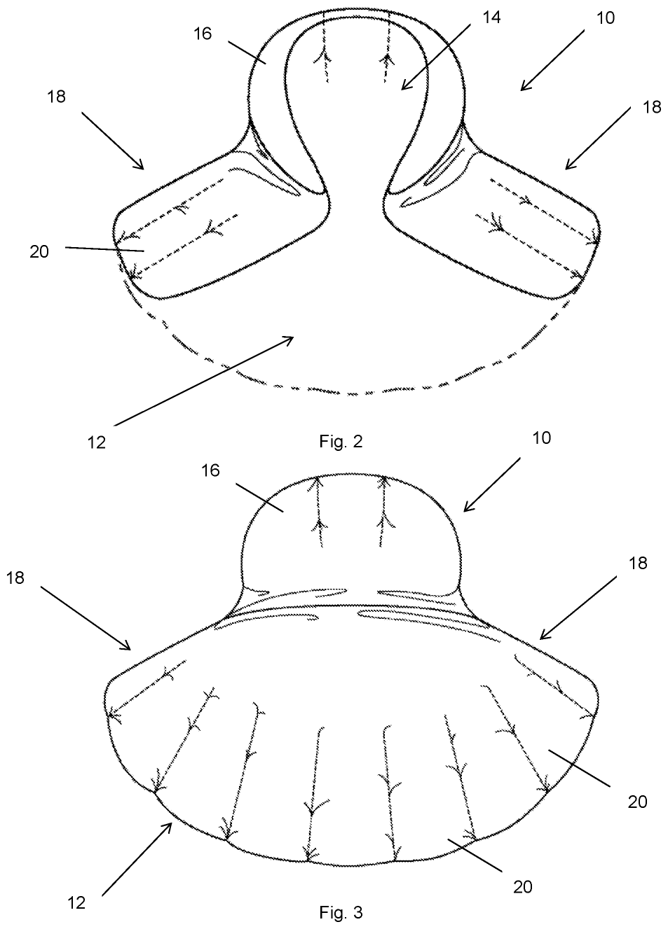

The blend of herbs and cannabis , and flax or other grain, is added to pouches that are formed into the aromatherapy garment. There are many different designs of aromatherapy garments into which the inventive blend described herein may be incorporated. One design is a neck and shoulder wrap as described in U.S. design Pat. D629,178, which has as its inventor the same inventor named herein and the entire disclosure of which is incorporated herein by this reference. With reference now to FIGS. 1 through 3, a neck and shoulder wrap aromatherapy garment 10 of the type that may be used to incorporate the compounds described herein, namely, blend of herbs and cannabis , and flax or other grain, is illustrated. Described generally, aromatherapy garment 10 is defined by a shawl-type garment that is designed to be worn around the user's neck, and which includes a back drape portion 12 that extends down the user's back, neck opening 14 that surrounds the user's neck and which includes an upwardly extending neck wrap portion 16 that conforms to the user's neck. Opposed shoulder portions 18 are located on opposite sides of the neck opening 14 and drape partly over the user's shoulders. With continuing reference to FIGS. 1 through 3, the aromatherapy garment 10 preferably includes plural internally-formed pouches 20 that are incorporated into the garment and into which the herb and cannabis and grain blends are packed.

It will be readily understood that in addition to the neck and shoulder wrap garment 10 illustrated in FIGS. 1 through 3, there are other aromatherapy garment designs into which the inventive blend of herbs and cannabis described herein may be incorporated include, but are not limited to, back wraps, lumbar wraps, body packs, breast packs, booties, comfort packs, mitts, neck packs, vests, neck wraps, eye pillows, sinus masks, sinus and migraine caps, and numerous other designs.

These and numerous other designs may be seen at applicant's websites at www. herbalconcepts.con, kozizones.com and www.cbdheatpack.com.

To use the wrap, the aromatherapy garment 10 is placed in a conventional microwave such as the kinds that are used in residential settings. Depending upon the power settings used, between 1 and 2 minutes of time in the microwave oven will heat the contents of the wrap sufficiently to cause vaporization of the essential oils. Not all of the essential oils and other compounds in the wrap vaporize at the same temperature. It has been found that the best results are obtained when the internal temperature of the herb blend in the pouches 20 has been heated to a temperature between about 150 to 165 degrees F. As described above, CBDA is decarboxylated to form CBD at a temperature of about 158 degrees F. Accordingly, when the aromatherapy wrap 10 according to the invention is heated to the desired temperature range just mentioned, CBDA that is contained in the herb blend is converted to CBD in the wrap so that CBD is released through the wrap to the user.

The aromatherapy garment used with the present invention defines a delivery vehicle for the essential oils and cannabinoid. Many different fabrics and materials may be used to fabricate the garment, including everything from hemp-based fabrics to polyesters, cottons, fake fur, muslin, cotton prints, silk, satins, fake snake skin, duck cloth, woven fabric and the like. Essentially, any fabric that can go into a microwave and which is strong enough to hold the contents may be used for the garment. It will be appreciated that the design of the garment and the material that is used to make it are not part of the present invention but act as the means of delivery for the volatilized compounds of interest.

Those of skill in the art will readily appreciate that invention described herein and illustrated in the drawings may be modified in certain manners to create equivalent embodiments without departing from the nature of the invention. As one example, the CBD content of the aromatherapy garment 10 may be replenished after it has been depleted by spraying a CBD-containing liquid blend onto the garment before or after the garment is heated in the microwave oven. A preferred replenishing blend contains approximately 33 mg/ml of CBD oil and may optionally include other essential oils such as lavender, lemon grass, peppermint or spearmint. In addition, the replenishing CBD-containing spray may be applied directly to the user's skin and the garment 10 used as usual.

The present invention has been described in terms of preferred and illustrated embodiments, it will be appreciated by those of ordinary skill that the spirit and scope of the invention is not limited to those embodiments, but extend to the various modifications and equivalents as defined in the appended claims.

1. Apparatus for delivering volatilized compounds, comprising:
a garment having internal an pouch;
a blend of phyto-derived materials contained in the pouch wherein the blend comprises a source of a cannabinoid.

2. The apparatus according to claim 1 in which the cannabinoid comprises CBD.

3. The apparatus according to claim 1 in which the cannabinoid comprises CBDA.

4. The apparatus according to claim 1 in which the cannabinoid comprises THC.

5. The apparatus according to claim 1 including a heat retention agent in the blend of phyto-derived materials.

6. The apparatus according to claim 5 in which the heat retention agent comprises a grain.

7. The apparatus according to claim 1 in which the source of the cannabinoid comprises between about 65 and 75 weight percent of the blend of phyto-derived materials.

8. The apparatus according to claim 7 wherein the source of the cannabinoid is sufficient to deliver between about 100 and 600 mg CBD.

9. A method for delivering volatilized compounds to a user, comprising the steps of:
a) providing a garment with an internal pouch and retaining in the pouch a blend of phyto-derived materials, including a source of a cannabinoid and other essential oils;
b) heating the garment to a temperature that is sufficient to volatilize the cannabinoid and other essential oils;
c) placing the heated garment in proximity to a user so that the user may inhale the volatilized cannabinoid and essential oils.

10. The method according to claim 9 including the step of adding a heat retention material to the blend of phyto-derived materials.

11. The method according to claim 10 in which the heat retention source comprises a grain.

12. The method according to claim 9 in which the cannabinoid comprises CBD.

13. The method according to claim 9 in which the cannabinoid comprises THC.

14. The method according to claim 9 in which the cannabinoid comprises CBDA.

15. The method according to claim 14 wherein the step of heating the garment comprises heating the garment to a temperature between about 150 and 165 degrees F. to thereby decarboxylate the CBDA.

16. The method according to claim 15 in which the garment is worn by the user so that decarboxlated CBDA and essential oils are absorbed through the user's skin and inhaled by the user.

17. The method according to claim 16 wherein the amount of decarboxylated CBDA available for absorption and inhalation is between about 100 and 600 mg.

18. The method according to claim 9 including the step of spraying onto the garment a liquid comprising a cannabinoid.

19. The method according to claim 17 in which the liquid further comprises selected volatile essential oils.

20. The method according to claim 9 in including the step of spraying onto the skin of the user a liquid comprising a cannabinoid prior to step c.
